(19) United States
(12) Patent Application Publication
(10) Pub. No.: US 2008/0091133 A1
(43) Pub. Date: Apr. 17, 2008

(54) Bandage with controlled environment

(76) Inventor: Jean Paul Matter, Indian Hills, OH (US)

Correspondence Address: William M. Selenke, 5 Drummond St., Cincinnati, OH, 45218, US

(21) Appl. No.: 11/580,257

(22) Filed: Oct. 12, 2006

(51) Int. Cl. A61F 15/00; A61F 13/00

(52) U.S. Cl. 602/41

(57) ABSTRACT

The invention is a bandage with a flat clear or opaque protective cover and a sidewall spacer attached to the outer circumference of the protective cover. The sidewall spacer has adhesives. This device has one or more inlet tubes and one or more outlet tubes and with monitoring sensors to monitor the bandaged wound environment. These sensors control pump(s) which provide liquids or gases that enter under the protective clear cover. The outlet tubes has an optional reversible closure. Likewise the bandage has a second cut through the protective cover for catheter chosen from of the group that includes urinary, dialysis, intravenous, intrarterial, total parental nutrition, or alimentary catheters.

### FIELD OF INVENTION

The present invention is concerned with coverings or bandages for injuries or wounds. Such injuries or wounds destroy the integrity of the skin that covers the human body. More specifically the present invention is concerned with controlling ambient air and therapeutic environment near the wound.

### BACKGROUND OF INVENTION

Human integument or skin is a remarkable organ. The skin has evolved to protect the internal environment of the living human being. Because of its vital function the skin needs to repair itself from the various cuts and scrapes which remove or damage the protective coating that is the skin. In ordinary environments and under ordinary conditions small cuts and bruises which damage the surface of the skin are readily repaired without concern.

Deeper cuts such as surgical incisions provide a different challenge. Muscles and tissues that are covered by the skin provide an excellent warm nutritious environment for bacterial growth. A surgical incision provides a direct path from the ambient environment into deeper tissues and organs. For this reason, surgical wounds and other breaks in the skin caused by accidents and so forth must be protected and monitored to promote more rapid healing.

In classical as well as contemporary practice, the standard treatment for various injuries and wounds is to cover them with sterile bandages, usually of cotton gauze or modern synthetic materials. Such bandages are inexpensive, easy to apply, and relatively useful in protecting the wound from bacteria in the ambient environment.

Likewise, gauze and other bandages can be useful in that they adsorb fluids oozing from the wound. However, the fluids that accumulate on the bandage provide a fertile environment for bacteria. Gauze bandages need to be removed to inspect the wound. Likewise, bandages can interfere with the healing process because the removal of the bandage can cause removal of the infiltrating cells that adhere to the bandage. Further a wound requires a certain degree of moisture to heal. However, for optimal healing a wound should not be excessively wet.

A number of processes break the integrity of the skin providing conditions to promote infection. Such skin trauma can be caused from either accidental cuts or intentional surgical procedures. For these reasons, there has been large body of art devoted to improvements in the basic gauze or cotton bandage. For example, U.S. Pat. No. 6,320,093 issued to Augustine et al. describes a bandage for autolytic debridement that is a fluid impermeable enclosure with a side wall to form a margin and a skin facing surface that surrounds the wound opening. His device is attached to the skin. The sidewall includes a fluid absorbent material. This bandage has the advantage that it can retain a humid environment and at the same time excess moisture or wound exudate is absorbed. Likewise the clear covering can allow physicians to observe the condition of the wound.

U.S. patent application 2004/0260253 which was published by Rosati, describe the bandage for supplying gases such as oxygen to target areas such as wounds.

U.S. Pat. No. 6,695,824 issued to Howard describes a wound dressing system combined with an irrigation system. This device helps remove excess moisture from wound and could be used to supply moisture or other fluids to a wound area. Rosati also teaches that his system provides a resealable opening through which one or more gases can be introduced into the dressing system and then resealed to prevent such gases from escaping.

U.S. Pat. No. 5,662,625 issued to Westwood describes a topical hyperbaric covering with a pressure control device to provide a constant high level of oxygen to an wound.

The elements of the present invention, as will be described below, provide a more nearly complete process for treating wounds that compromise the integrity of the skin. That is, the present invention provides a clear surface to monitor the wound. It likewise is provides with positive pressure from an environmental pump which controls the amount of the gases and oxygen level in the environment of the wound. which positive pressure will urge the cover from the wound and skin. The present invention will provide an optimal level of oxygen and water vapor which may be oxygenated to promote healing or may be anoxic to promote angiogenesis. If desired, antibiotics and or other medicaments can be placed on the wound without removing the bandage. Likewise, a limited amount of wound surface cleaning can be done by introducing cleaning liquids into the covered space. The clear protective cover bandage like device to cover and to provide an optimal environment for wound healing may be modified to be opened and resealed to surround an indwelling catheter that passed through the protective cover which provides an environment to surround the wound created by an indwelling catheter. Indwelling catheters may be urinary, dialysis, intravenous, intrarterial, total parental nutrition, or alimentary.

### SUMMARY OF INVENTION

The invention is a device to cover and provide an optimal environment for wound healing It has a flat clear or opaque protective cover and a sidewall spacer attached to the outer circumference of the protective cover. The sidewall spacer has an attachment means so that device with protective cover can be affixed to skin surrounding the wound. This device has one or more inlet tubes and one or more outlet tubes and an optional pressure regulator on one or more of the outlet tubes. The device has one or more and monitoring sensors to monitor the environment defined by the sidewall spacer attached to the outer circumference of the clear protective cover. These sensors control pump(s) which provide fluids that enter under the protective clear cover. At least one of the outlet tubes has a reversible closure. The device to cover and provide an optimal environment The device to cover and provide an optimal environment for wound healing has a second cut through said clear protective cover for catheter chosen from of the group that includes urinary, dialysis, intravenous, intrarterial, total parental nutrition, or alimentary catheters.

### BRIEF DESCRIPTION OF FIGURES

FIG. 1 shows a bandage like device to cover and to provide an optimal environment for wound healing.

FIG. 2 shows a section cut of the invention.

FIG. 3 shows a bandage like device to cover and to provide an optimal environment for reducing infection caused by an indwelling catheter.

### DETAILED DESCRIPTION OF FIGURES

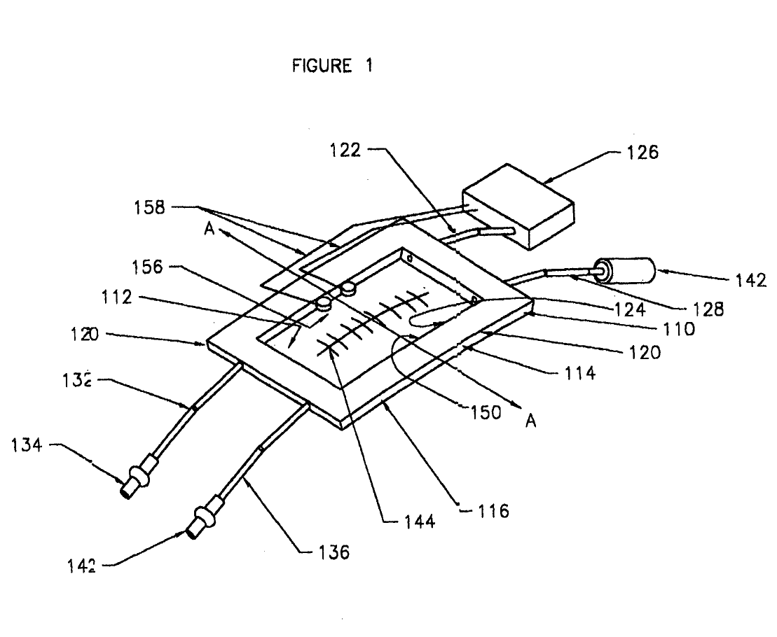

FIG. 1 shows a bandage like device 110 to cover and to provide an optimal environment for wound healing. A sidewall spacer 114 is attached to the outer circumference 120 of clear optionally opaque protective cover 112. (A sutured surgical wound 144 [in outline] is shown covered and protected by the device.). Sidewall spacer 114 has a reversible attachment means such as adhesives 116 so that the protective cover can be affixed to surround the wound;

A first inlet tube 122 passes into the environment 124 defined by sidewall spacer 114 and clear protective cover 112. First inlet tube 122 originated and transfers fluids from monitoring workpiece pump 126 which provides a controlled gas source A second preferably larger inlet tube 128 with a closeable end cap 142 may be used to introduce irrigating fluids or medicaments such as antibiotics into the environment 124 defined by patient's skin 150, sidewall spacer 114, and clear protective cover 112.

A first outlet tube 132 has a regulator 134 to provide back pressure to assure a positive pressure on the environment 124 defined by patient's skin 150, sidewall spacer 114, and clear protective cover 112. A second outlet tube 136, preferably larger, provides an outlet for fluids introduced through inlet tube 128 with a closeable end cap 142. Second outlet tube 136 a closeable second end cap 142. Such fluids may be introduced for cleaning and therapeutic purposed into the environment 124 defined by sidewall spacer 114 and clear protective cover 112. One or more sensors 156 to monitor the environment of the wound determine and communicates 158 such needed information to monitoring workpiece pump 126 which is microprocessor controlled. The monitoring workpiece pump 126 introduce proper fluids via first inlet tube 122 into environment 124 defined by sidewall spacer 114 and clear protective cover 112. The benefits of the bandage like device 110 is that it can cover and provide an optimal environment for wound healing. At the same time, the physician can monitor the wound without removing the bandage. Such removal of the bandage may interfere with healing. Another benefit is that cleaning solution with antibiotics and other such medicaments can be introduced to treat the wound without removing the bandage.

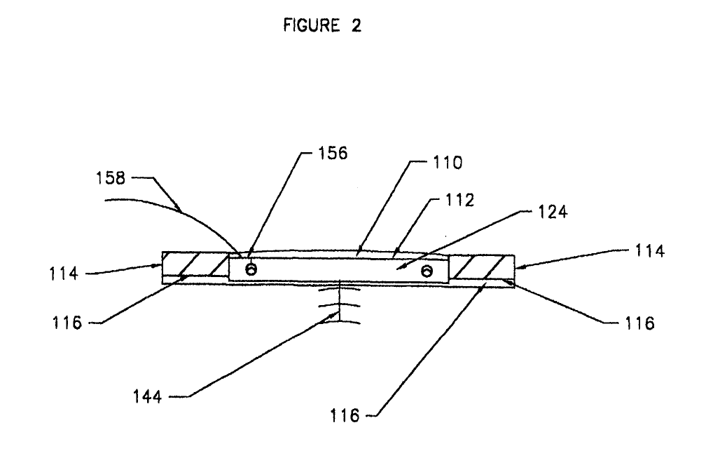

FIG. 2 shows a section shown by cut AA in FIG. 1. Bandage like device 110 to cover and to provide an optimal environment for wound healing. A sidewall spacer 114 is attached to the outer circumference 120 of clear protective cover 112 for patient's skin 150. (A sutured surgical wound 144 [in outline] is shown covered and protected by the device.). Sidewall spacer 114 has a reversible attachment means such as adhesives 116 so that the protective cover can be affixed said protective cover can be affixed to skin surrounding the workpiece wound 144. Sensor 156 to monitor the environment of the wound determine and communicates 158 such needed information to monitoring workpiece pump 126.

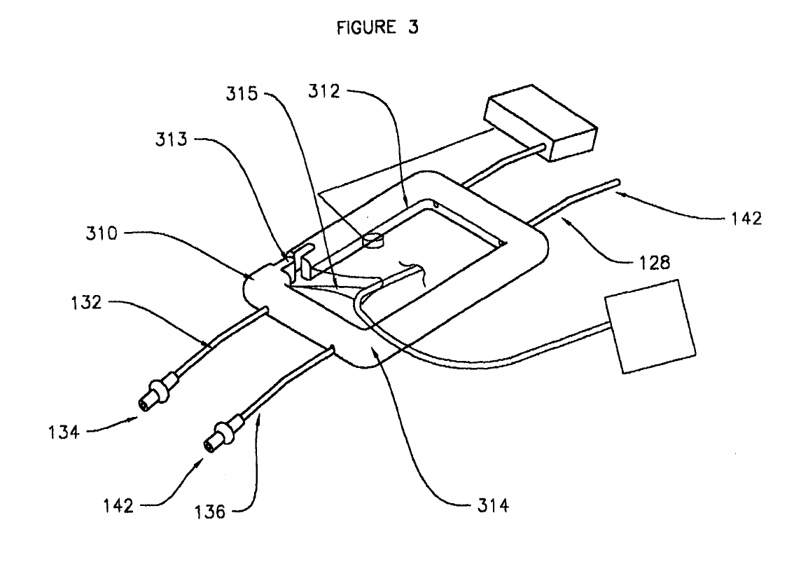

FIG. 3 shows a bandage like device 310 to cover and to provide an optimal environment for reducing infection caused by an indwelling catheter. A sidewall spacer 314 is attached to the outer circumference 320 of clear or opaque protective cover 312. A first cut 313 through sidewall spacer 314 proceeds as second cut 315 through clear protective cover 312. Both first cut 313 and second cut 315 are designed to overlap and reseal with the indwelling catheter passing through. Sidewall spacer 314 has a reversible attachment means such as adhesives 116 so that said protective cover can be affixed to surround the wound.

I claim:

1) A device to cover and provide an optimal environment for wound healing comprising;
a flat clear or opaque protective cover;
a sidewall spacer attached to the outer circumference of said protective cover;
said sidewall spacer has an attachment means so that said protective cover can be affixed to skin surrounding the wound;
one or more inlet tubes;
one or more outlet tubes.

2) A device to cover and provide an optimal environment for wound healing as in claim 1) wherein said device has one or more and monitoring sensors to monitor the environment defined by said sidewall spacer attached to the outer circumference of said protective cover;
said sensors control the pumps which provide fluids that enter said protective cover.

3) A device to cover and provide an optimal environment for wound healing as in claim 1) wherein at least one of said outlet tubes has a reversible closure.

4) A device to cover and provide an optimal environment for wound healing as in claim 1) wherein said affixable means is an adhesive.

5) A device to cover and provide an optimal environment for wound healing as in claim 1) wherein at least one of said outlet tubes has a regulator.

6) A device to cover and provide an optimal environment for wound healing as in claim 1) wherein at least one of said inlet tubes is connected to the controlled gas source.

7) A device to cover and provide an optimal environment for wound healing comprising; a flat clear or opaque protective cover;
the sidewall spacer attached to the outer circumference of said protective cover;
a first cut which proceeds said sidewall spacer and continues as a second cut through said protective cover;
both said first cut and said second cut passing through said clear protective to cover, overlap, and reseal an indwelling catheter.
said sidewall spacer has an attachment means so that said protective cover can be affixed to skin surrounding the wound;
one or more inlet tubes;
one or more outlet tubes.

8) A device to cover and provide an optimal environment for wound healing as in claim 7) wherein said device has one or more and monitoring sensors to monitor the environment of the wound;
said sensors control the pumps which provide fluids that enter said protective clear cover.

9) A device to cover and provide an optimal environment for wound healing as in claim 7) wherein at least one of said outlet tubes has a reversible closure.

10) A device to cover and provide an optimal environment for wound healing as in claim 7) wherein said affixable means is an adhesive.

11) A device to cover and provide an optimal environment for wound healing as in claim 7) wherein at least one of said outlet tubes has a pressure regulator.

12) A device to cover and provide an optimal environment for wound healing as in claim 7) wherein at least one of said inlet tubes is connected to the controlled gas source.

13) A device to cover and provide an optimal environment for wound healing as in claim 7) wherein said catheter is one of the group that includes urinary, dialysis, intravenous, intrarterial, total parental nutrition, or alimentary catheters.
